(19) United States
(12) Patent Application Publication
(10) Pub. No.: US 2025/0127500 A1
(43) Pub. Date: Apr. 24, 2025

(54) SEPTAL MYECTOMY RETRACTOR

(71) Applicant: University of Florida Research Foundation, Incorporated, Gainesville, FL (US)

(72) Inventor: Susan Amanda COOKE, Gainesville, FL (US)

(21) Appl. No.: 18/684,982

(22) PCT Filed: Aug. 22, 2022
(86) PCT No.: PCT/US2022/041081

§ 371 Date: Feb. 20, 2024

Related U.S. Application Data
(60) Provisional application No. 63/235,442, filed on Aug. 20, 2021.

(51) Int. Cl. A61B 17/02; A61B 17/3211

(52) U.S. Cl. CPC A61B 17/0218; A61B 17/3211; A61B 2017/0225; A61B 2017/0237

(57) ABSTRACT

Disclosed is a surgical instrument that includes a retractor that is adjustable in 1 or 2-dimensions and a handle with a body having a distal end, a proximal end, and at least one channel inside the body. The handle is connected to the retractor at the proximal end by two wires inside the channel, a stationary and a dynamic wire. The dynamic wire is linked, directly or indirectly, to an actuator such that actuation of the actuator moves the dynamic wire relative to the retractor. As the dynamic wire moves in a first direction, the retractor widens, and may optionally shorten. The retractor has a flexible mesh through which the dynamic wire extends. Also disclosed is a method for a septal myectomy using the surgical instrument disclosed within.

### BACKGROUND

Hypertrophic Obstructive Cardiomyopathy (HOCM) is a genetic defect that occurs in 1 in 500 people in the United States. There are 113 Congenital Hear Centers across the United States, and it is estimated that HOCM affects 20 million people worldwide. Septal myectomy is a procedure to remove the enlarged tissue from the septum in patients with HOCM. A septal myectomy is complicated by complete heart block or injury to the aortic valve leaflets that leave the valve incompetent. The current retractors used in the procedure are often inadequate, create difficulties in visualization, and require frequent adjustments, which have the potential of injuring the aorta, the aortic valve leaflets, and conduction tissue depending on pressure and angle of retraction.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1 shows a side perspective view of a surgical instrument comprising an expandable retractor.

FIG. 2 shows a diagram showing a method of use of the embodiments shown in FIG. 1.

FIG. 3A shows a side perspective view of another instrument embodiment. FIG. 3B shows close-up view of a portion the instrument embodiment shown in FIG. 3A.

FIG. 4A shows a side perspective view of another instrument embodiment in a non-expanded state. FIG. 4B shows a side perspective view of the instrument in FIG. 4A in an expanded state.

### DEFINITIONS

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated container and method, and such further applications of the principles of the invention as illustrated therein being contemplated as would normally occur to those skilled in the art to which the invention pertains.

It is to be noted that the terms “first,” “second,” and the like as used herein do not denote any order, quantity, or importance, but rather are used to distinguish one element from another, unless otherwise specifically stated herein. The terms “a” and “an” do not denote a limitation of quantity, but rather denote the presence of at least one of the referenced item. The modifier “about” used in connection with a quantity is inclusive of the stated value and has the meaning dictated by the context. The term “about” generally refers to a range of numbers that one of skill in the art would consider equivalent to the recited value (i.e., having the same function or result). In a specific embodiment, the term “about” includes a stated numerical value as well as a value that is +/−30% of the stated numerical value. For example, about 40 degrees includes 40 degrees as well as angles of 36 degrees and 44 degrees, and all values in between. In many instances, the term “about” may include numbers that are rounded to the nearest significant figure. Furthermore, to the extent that the terms “including,” “includes,” “having,” “has,” “with,” or variants thereof are used in either the detailed description and/or the claims, such terms are intended to be inclusive in a manner similar to the term “comprising.”

It is to be noted that all ranges disclosed within this specification are inclusive and are independently combinable. Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. As a non-limiting example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 7.

As used herein, the terms “subject”, “user” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human.

### DETAILED DESCRIPTION

Conventional septal myectomy retractors have several drawbacks. They do not protect the entire valve and require constant readjusting. The pressure applied to the valves depends on the assistant's hands/expertise which can lead to slips. This can injure the aortic valve, aorta, and conduction. These problems lead to longer surgeries and recovery time for the patients.

Provided is a septal myectomy retractor that reduces the need to adjust by stenting the aorta open, enabling better and constant visualization. The diameter of the retractor can be adjusted to the known aortic valve annulus. The retractor covers and protects leaflets from potential harm as the surgeon enters and exits the aorta with sharp instruments The retractor can significantly increase the efficiency and speed of the operation particularly reducing the crossclamp time of the operation resulting in a shorter period of myocardial ischemia.

According to one embodiment, provided is a surgical instrument that includes a retractor that is adjustable so as to be radially expandable and a handle with a body having a distal end, a proximal end, and, optionally, at least one channel inside the body. In one embodiment, the handle is connected to the retractor at the distal end by two wires inside the channel, a stationary and a dynamic wire. The handle has an actuator that when actuated drives the expansion of the retractor. In a specific embodiment, the actuator rotates relative to the proximal end of the body, and the rotating wire is interlinked, either directly or indirectly, to the actuator such that rotation of the actuator causes the dynamic wire to extend from the distal end of the surgical instrument.

In another specific embodiment, the retractor is adjustable in two-dimension, length and diameter. In an even more specific embodiment, as the dynamic wire moves in a first direction, the retractor shortens and widens. When the dynamic wire moves in a second direction, the retractor narrows and lengthens. The first direction is actuated by a counterclockwise turning of the actuator relative to the body, and the second direction is actuated by a clockwise turning of the actuator relative to the body, or vice versa. In a specific embodiment, the actuator has a diameter of about 17 mm to 19 mm, and the actuator is a dial that clicks when it turns, for example about 0.1-1.5 mm per click.

In a specific embodiment, the retractor is about 5 to 6 cm long when compressed and about 3 to 4 cm long when expanded. As the size of the aortic root varies based on patient body size and heart disease, the expected range from collapsed state to expanded state of the retractor is about 11 mm to about 35 mm. When expanded, the retractor is about 1.5-3 mm wider then when compressed. In a specific embodiment, the retractor adjusts from about 17 mm to 19 mm, i.e., about 17 mm wide when compressed and about 19 mm wide when expanded. In other embodiments, the retractors may be configured to have an outward expansion (width or diameter) range, including, but not limited to, about 11 mm to 13 mm, about 13 mm to 15 mm, about 15 mm to 17 mm, about 17 mm to 19 mm, about 19 mm to 21 mm, about 21 mm to 23 mm, about 23 mm to 25 mm, about 25 mm to 27 mm, about 27 mm to 29 mm, about 29 mm to 31 mm, about 31 mm to 33 mm, and about 33 mm to 35 mm.

According to another embodiment, disclosed is a surgical instrument for performing a septal myectomy on a patient in need that includes a retractor comprising a wall that defines an inner conduit, the retractor being adjustable in at least one or two dimensions (such as width, diameter, circumference, and length). The retractor can take one of many forms, but in typical embodiments, the retractor is generally tubular. The instrument includes an elongated handle comprising a body comprising a distal end and a proximal end. Associated with the handle body is an actuator, typically positioned at or near the proximal end of the body. The handle body may also include at least one channel defined therein. The instrument includes a stationary wire associated with the handle and the retractor, wherein a portion of the stationary wire extends around at least a portion of the retractor. The surgical instrument also includes a dynamic wire linked directly or indirectly to the actuator, such that when the actuator is actuated, the dynamic wire moves relative to the retractor to adjust the retractor in the at least one or two dimensions. In one embodiment, the dynamic wire moves within the conduit upon actuation of the actuator such that the dynamic wire applies outward force on the wall. In a specific embodiment, the dynamic wire moves distally within the conduit in a spiral fashion down through the conduit upon actuation of the actuator. In an alternative embodiment, the dynamic wire is comprised of different components. For example, the dynamic wire may include a plurality of wire channel segments through which a continuous wire is disposed. As force is applied to the dynamic wire, such as via the continuous wire, the plurality of segments spread apart causing an expansion of the retractor.

Thus, according to a specific embodiment, provided is a surgical instrument for performing a septal myectomy on a patient in need comprising: a retractor comprising a wall that defines an inner conduit, the retractor being adjustable in at least one or two-dimensions and an elongated handle comprising a body comprising a distal end and a proximal end, an actuator, and, optionally, at least one channel defined in said body, a stationary wire associated with the handle and the retractor, wherein a portion of the stationary wire extends around at least a portion of the retractor, and a dynamic wire linked directly or indirectly to the actuator, such that when the actuator is actuated the dynamic wire moves relative to the retractor to adjust the retractor in the at least one or two dimensions. The dynamic wire is comprised of a plurality of segments and a continuous wire that runs therethrough. The end of the dynamic wire comprises an end-cap. As force is applied to the dynamic wire, the plurality of segments separate causing radial expansion of the retractor.

In a related alternative embodiment, the instrument may comprise at least one flexible wire channel defined in the wall through which the dynamic wire moves. The at least one wire channel may also comprise a closed distal end upon which the dynamic wire ultimately abuts. In a specific embodiment, the wire channel spirals distally along the wall of the retractor. Similar to the multicomponent dynamic wire described in the preceding paragraph, as force is applied to the dynamic wire and pushes against the closed distal end, the at least one wire channel flexes outwardly with the wall of the retractor.

In certain embodiments, the stationary wire extends up to halfway around the perimeter of the retractor. Typically, the stationary wire is positioned at the proximal end of the retractor.

In certain embodiments, the retractor reversibly adjusts from a first state having a first length and a first width to a second state having a second length and a second width, wherein the first length is the same or greater than the second length, and the second width is greater than the first width. In a specific embodiment, the retractor comprises a length of about 5-6 cm when in the first state, and about 3-4 cm when in the second state. In a specific embodiment, the retractor comprises a width of about 1-3 mm, or about 2 mm, greater when in the second state compared to the first state.

In a specific embodiment, the wall of the retractor comprises a flexible mesh. Those skilled in the art will appreciate that the flexible mesh can be made of any suitable material. In a specific embodiment, the flexible mesh is comprised of metal. The flexible mesh may also be coated with a biosafe material, such as, but not limited to, silicone or plastic.

In certain embodiments, the handle comprises a channel, wherein at least a portion of the stationary wire and at least a portion of the dynamic wire are disposed in the channel. In a specific embodiment, the dynamic wire links directly or indirectly to the actuator through the channel. Alternatively, the dynamic wire links directly or indirectly to the actuator outside the channel.

The surgical instrument may also be coated with a biosafe material such as silicone. This coating will protect the aortic valve leaflets from the wire mesh.

According to another embodiment, disclosed is method for a septal myectomy in a patient in need starting with performing a median sternotomy by making an incision down the middle of a patient's chest and separating the breastbone. The patient is put on cardiopulmonary bypass (CPB) and a crossclamp is placed across the aorta. Once cardiac arrest is achieved a transverse aortotomy is made by the surgeon. The method includes inserting the surgical instrument embodiment described herein into a patient's aorta. The surgical instrument in inserted into the aortic root pushing the aortic valve leaflets away from center. The surgical instrument is then actuated to expand to a previously known diameter or width specific to the patient. The surgeon then repeatedly inserts a scalpel through the surgical instrument into a left ventricle, cutting a section of a left ventricle septum, and removing the scalpel and section of left ventricle septum. Finally, the method includes dialing the surgical instrument back to its compressed state removing the surgical instrument, closing the aortotomy, removing the crossclamp, weaning from CPB, closing the breastbone and the patient's chest.

Another method for a septal myectomy in a patient in need includes the steps of obtaining access to a patient's heart, inserting the retractor of the surgical instrument of one of the disclosed embodiments into a patient's aortic root, expanding the retractor, inserting a cutting instrument through the surgical instrument into a left ventricle, cutting a section of a left ventricle septum, removing the cutting instrument and section of left ventricle septum, contracting the surgical instrument, removing the surgical instrument, and closing access to the patient's heart.

### Description of Illustrated Embodiments

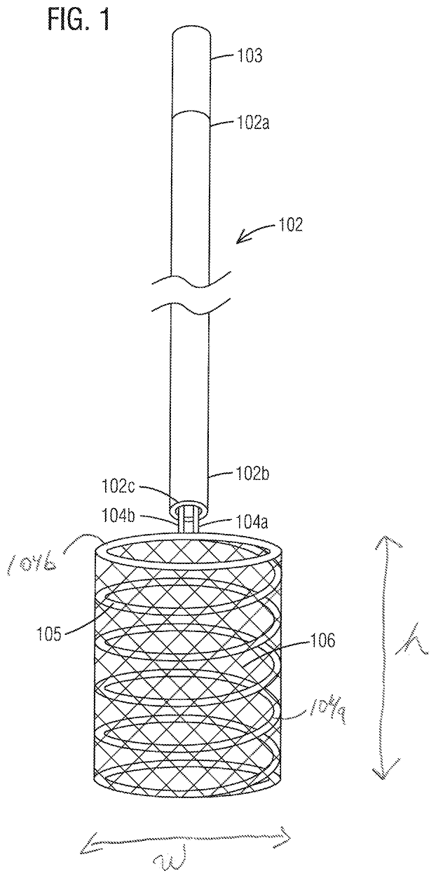

Turning to the drawings, FIG. 1 shows a side perspective view of a medical instrument embodiment 100. The medical instrument 100 includes an elongated handle 102 with a proximal end 102a, a distal end 102b, and a channel 102c defined within the handle 102. In specific embodiments, the elongated handle 102 is about 17.5 cm long. Associated with the proximal end 102a is an actuator 103 that moves relative to the elongated handle 102. The actuator can take multiple different forms, such as, but not limited to, a dial, knob or button. As shown in FIG. 1, the actuator is a dial. In certain embodiments, the actuator 103 has an expandable width (w) mm and, optionally an adjustable height (h). In a specific embodiment, the actuator is a dial that clicks as it turns, wherein each click may represent a predetermined expansion of a retractor 105. In an even more specific embodiment, each click represents 0.1-2 mm per click. The actuator 103 interlinks, either directly or indirectly, to two wires 104a,b. As shown, the wires 104a and 104b extend out of the distal end 102b through the channel 102c. The two wires include a dynamic wire 104a and a stationary wire 104b. The actuator 103 controls the movement of the dynamic wire 104a. In alternative embodiments, the actuator 103 is linked with the dynamic wire 104a either directly or by an indirect mechanism positioned outside the channel 102c.

As the actuator 103 is actuated, the dynamic wire 104a is directed into the retractor 105 causing the outward radial expansion of the retractor 105. In a specific embodiment, the retractor 105 includes a flexible mesh 106. Those skilled in the art will appreciate that the flexible mesh 106 can be made of any suitable biosafe materials, such as polymers or metal, so long as it is made of a material possessing sufficient flexibility to allow the flexible mesh 106 to expand in response to movement of the dynamic wire 104a. In a specific embodiment, the retractor 105 may be about 3 to 6 cm long, about 17 mm wide when in non-expanded state, and about 19 mm wide when in an expanded state. The center of the retractor 105 is hollow or open to allow for other surgical instruments to pass through.

In certain embodiments, the actuator is interlinked with a mechanism to apply rotary motion such that the dynamic wire rotates out of the distal end of the handle and into the retractor. Alternatively, the mechanism converts linear motion into rotary motion, or rotary motion into linear motion. The mechanism is included in the channel of the handle.

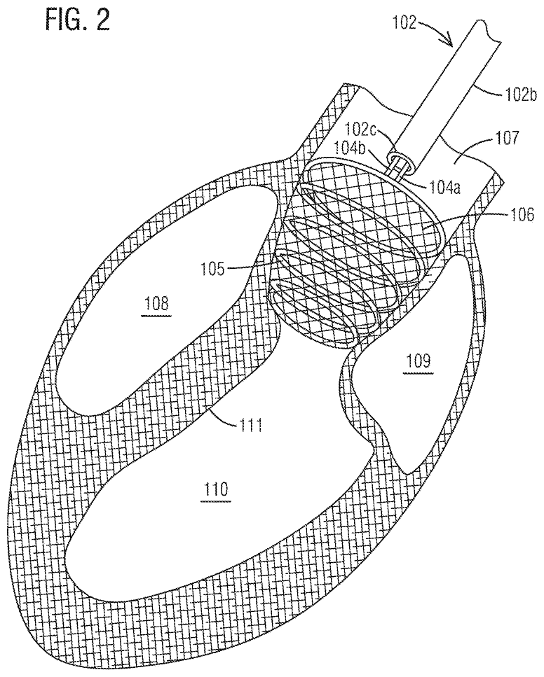

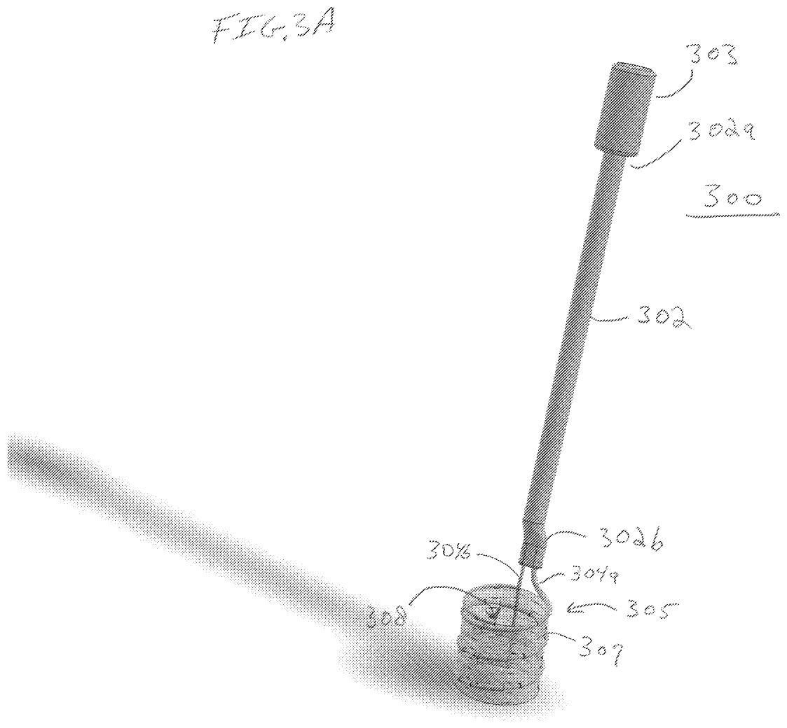

FIG. 2 shows a side perspective view of a method for a septal myectomy in a patient using medical instrument embodiment 100. After a surgeon or medical professional has obtained access, the retractor 105 is inserted into a patient's aorta. For reference and not drawn to scale, the patient's right atrium 108 and left atrium 109 are also shown. The medical professional actuates the actuator 103 to expand the retractor 105 allowing for a scalpel or cutting instrument to pass through the retractor 105 and aorta 107 into the patient's left ventricle 110. The medical professional cuts the hypertrophied left ventricle septum 111 and removes it and the cutting instrument. Finally, the rotating segment 103 is turned in the opposite direction to collapse the retractor 105, and the medical instrument is removed from the patient's aorta 107.

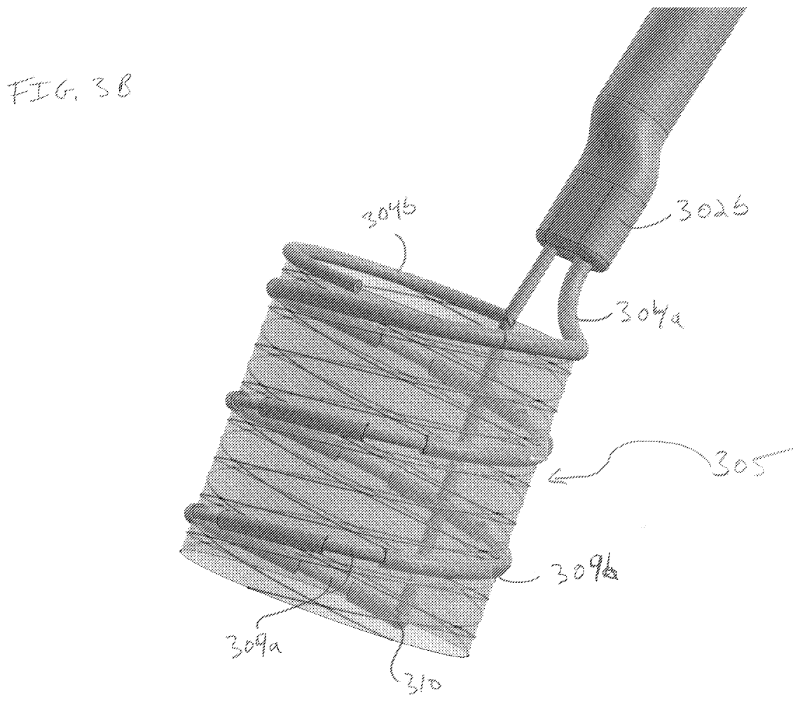

FIG. 3A shows a side perspective view of another surgical instrument embodiment 300. The instrument 300 includes an elongated handle 302 with a proximal end 302a, a distal end 302b, and a channel (not shown) defined within the handle 302. Associated with the proximal end 302a is an actuator 303 that moves relative to the elongated handle 302. As with actuator 103, actuator 303 can take multiple different forms, such as, but not limited to, a dial, knob or button. As shown in FIG. 3, the actuator is a rotating knob. Associated with the distal end 302b is a retractor 305. Wires 304a and 304b extend out of the distal end 302b. Wire 304a is a dynamic wire and wire 304b is a stationary wire. The stationary wire 304b may flex but is not intended to extend or retract. In this embodiment of the instrument 300, a portion of the stationary wire 304b extends around a portion of the proximal end of the retractor 305. The retractor 305 comprises a wall 307 that defines a conduit 308.

FIG. 3B shows a close up view of the distal end 302b and the retractor 305. In this alternative embodiment, the dynamic wire 304a is comprised of different dynamic components. The dynamic wire 304a comprises a continuous wire 309a and plurality of segmented channels 309b into which the continuous wire 309a is disposed. At the end of the dynamic wire 304a is an end-cap 311. Also shown is a stability bar 312 connected to, or integrated as part of, the stationary wire 304b. Actuation of the actuator 303 controls expansion of the retractor 305 by creating a force applied to the dynamic wire (e.g. via the continuous wire 309a) thereby causing an separation of the segmented channels 309b. The extension of the dynamic wire 304a is reversible by the actuator 303. Thus, the retractor 305 can reversibly adjust from a first state having a first width to a second state having a second width, wherein the second width is greater than the first width. As with actuator 103, actuator 303 can be linked with the dynamic wire 304a either directly or by an indirect mechanism positioned outside the channel 302c.

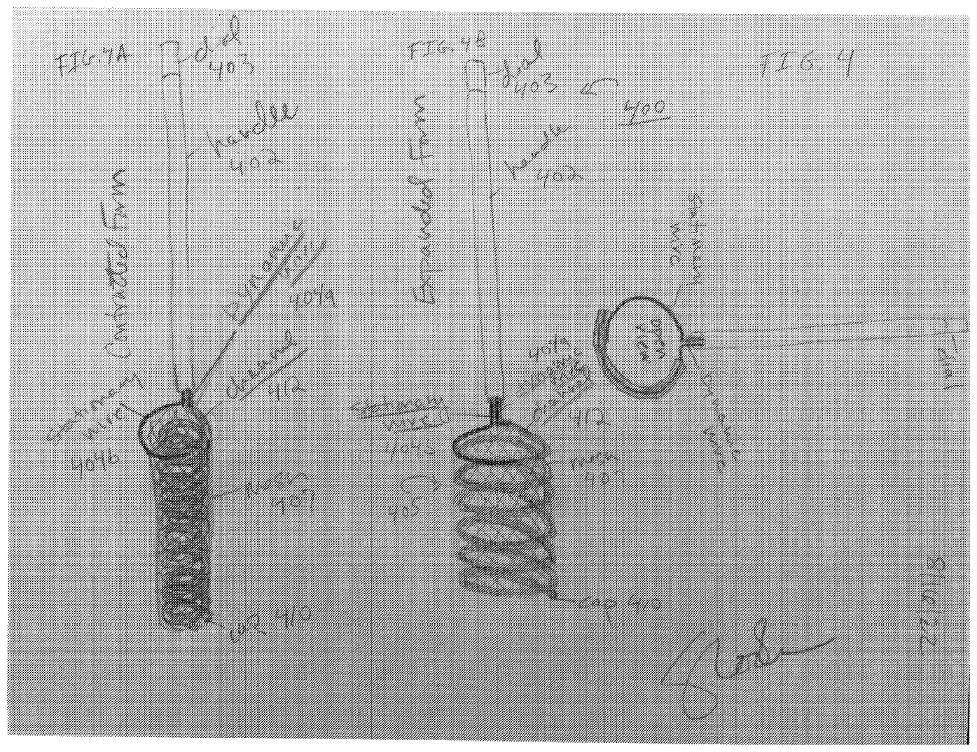

Turning to FIG. 4, shown is another version of a surgical instrument 400. The instrument 400 comprises a handle 402 and an actuator 403. The instrument 400, also includes a retractor 405 with a mesh wall 407 and a wire channel 412 that is disposed along the wall 407 of the retractor 405, and the wire channel 412 has a closed end 410. The wire channel 412 may be able to stretch or flex, or otherwise allow for radial expanded movement. In such embodiment, the dynamic wire 404a involves a wire that courses through the wire channel 412 and the force of the dynamic wire 404a in the wire channel 412 causes the expansion of the retractor.

While one or more embodiments of the present invention have been shown and described herein, such embodiments are provided by way of example only. Variations, changes and substitutions may be made without departing from the invention herein. Accordingly, it is intended that the invention be limited only by the spirit and scope of the appended claims. The teachings of all references cited herein are incorporated in their entirety to the extent not inconsistent with the teachings herein. Alternatives, Deviations and modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

What is claimed is:

1. A surgical instrument for performing a septal myectomy on a patient in need comprising: a retractor comprising a wall that defines an inner conduit, the retractor being adjustable in at least one or two-dimensions and an elongated handle comprising a body comprising a distal end and a proximal end, an actuator, and, optionally, at least one channel defined in said body, a stationary wire associated with the handle and the retractor, wherein a portion of the stationary wire extends around at least a portion of the retractor, and a dynamic wire linked directly or indirectly to the actuator, such that when the actuator is actuated the dynamic wire moves relative to the retractor to adjust the retractor in the at least one or two dimensions.

2. The surgical instrument of claim 1, wherein the dynamic wire moves within the conduit upon actuation of the actuator such that the dynamic wire applies outward force on the wall.

3. The surgical instrument of claim 1 or 2, wherein the retractor is generally tubular.

4. The surgical instrument of any of claims 1-3, wherein dynamic wire moves distally within the conduit in a spiral fashion down through the conduit upon actuation of the actuator.

5. The surgical instrument of any of claims 1-4, wherein the retractor further comprises at least one wire channel defined in the wall through which the dynamic wire moves.

6. The surgical instrument of claim 5, wherein the at least one wire channel comprises a closed distal end upon which the dynamic wire abuts.

7. The surgical instrument of any of claims 1-6, wherein the one or two dimensions are selected from width, diameter, circumference, and length.

8. The surgical instrument of any of claims 1-7, wherein the retractor reversibly adjusts from a first state having a first length and a first width to a second state having a second length and a second width, wherein the first length is the same or greater than the second length, and the second width is greater than the first width.

9. The surgical instrument of any of claims 1-8, wherein the handle comprises a channel and at least a portion of the stationary wire and at least a portion of the dynamic wire are disposed in the channel.

10. The surgical instrument of any of claims 1-8, wherein the dynamic wire links directly or indirectly to the actuator through the channel.

11. The surgical instrument of any of claims 1-8, wherein the dynamic wire links directly or indirectly to the actuator outside the channel.

12. The surgical instrument of any claims 8-11, wherein the retractor comprises a length of about 5-6 cm when in the first state, and about 3-4 cm when in the second state.

13. The surgical instrument of any of claims 1-12, wherein the actuator is a dial, knob or button.

14. The surgical instrument of claim 13, wherein the actuator comprises a rotating segment that rotates relative to the proximal end of the body, wherein the dynamic wire is linked directly or indirectly to the rotating segment such that rotation of the rotating segment moves the wire relative to the retractor.

15. The surgical instrument of any of claims 1-14, wherein the instrument comprises a mechanism to translate linear motion into rotary motion, or rotary motion to linear motion.

16. The surgical instrument of claim 5 or 6, wherein the wire channel spirals distally along the wall and comprises a closed distal end.

17. The surgical instrument of any of claims 1-16, wherein the stationary wire extends up to halfway around the perimeter of the retractor.

18. The surgical instrument of claim 17, wherein the stationary wire is positioned at the proximal end of the retractor.

19. The surgical instrument of any of claims 1-18, wherein the actuator is a rotating segment that comprises a diameter of about 11 mm to 35 mm.

20. The surgical instrument of claim 19, wherein the rotating segment clicks when turned, and turns about 0.1-1.5 mm per click.

21. The surgical instrument of any of claims 1-20, wherein the wall of the retractor comprises a flexible mesh.

22. The surgical instrument of claim 21, wherein the mesh is comprised of metal.

23. The surgical instrument of claim 21 or 22, wherein the mesh is coated with a biosafe material.

24. The surgical instrument of claim 19, wherein the dynamic wire moves in a first direction by a counterclockwise turning of the rotating segment relative to the body, and the dynamic wire moves a second direction by a clockwise turning of the rotating segment relative to the body, or vice versa.

25. The surgical instrument of claim 8, wherein the retractor comprises a length of about 5 to 6 cm long when in the first state and about 3 to 4 cm when in the second state.

26. The surgical instrument of claim 8, wherein the retractor comprises a width of about 1-3 mm, or about 2 mm greater when in the second state compared to the first state.

27. The surgical instrument of claim 23, wherein the biosafe material comprises silicone.

28. A method for a septal myectomy in a patient in need comprised obtaining access to a patient's heart, inserting the retractor of the surgical instrument of any of claims 1-27 into a patient's aortic root, expanding the retractor, inserting a cutting instrument through the surgical instrument into a left ventricle, cutting a section of a left ventricle septum, removing the cutting instrument and section of left ventricle septum, contracting the surgical instrument, removing the surgical instrument, and closing access to the patient's heart.
